(19) United States
(12) Patent Application Publication
(10) Pub. No.: US 2020/0140889 A1
(43) Pub. Date: May 7, 2020

(54) SELECTABLE STEM CELL MODIFICATION FOR GENE THERAPY USING TRANSIENT CELL SURFACE MARKING OF MODIFIED CELLS USING MODIFIED CD4 MOLECULE

(71) Applicant: TEXAS TECH UNIVERSITY SYSTEM, Lubbock, TX (US)

(72) Inventors: Himanshu GARG, El Paso, TX (US); Anjali JOSHI, El Paso, TX (US)

(21) Appl. No.: 16/607,710

(22) PCT Filed: Apr. 24, 2018
(86) PCT No.: PCT/US2018/029213

§ 371 Date: Oct. 23, 2019

Related U.S. Application Data
(60) Provisional application No. 62/488,972, filed on Apr. 24, 2017.

(51) Int. Cl. C12N 15/86; C12N 15/11; C12N 15/10; G01N 33/543; C07K 14/73

(52) U.S. Cl. CPC C12N 15/86; C12N 15/11; C12N 15/102; C12N 2310/20; C07K 14/70514; C12N 2740/15043; G01N 33/54326

(57) ABSTRACT

Aa gene therapy method comprises modifying at least one CD4 molecule to form CD4mod, marking target cells with immunomagnetic beads, and killing the marked target cells. The method comprises applying a first vector that expresses TK-SR39 and applying a second vector that expresses HIV Tat and a CRISPR-CCR5 cassette to knockout CCR5. The TK-SR39 rapidly kills cells in a presence of Ganciclovir.

### TECHNICAL FIELD

Embodiments generally relate to gene therapy.

Embodiments further relate to gene therapy for selectable stem cell modification thus providing gene therapy using transient cell surface marking of modified cells using a modified CD4 molecule.

Embodiments also relate to gene therapy for selectable stem cell modification thus providing anti-HIV gene therapy using transient cell surface marking of modified cells using a modified CD4 molecule.

Embodiments further relate to modified CD4 as a cell surface marker that can be used to purify vector transduced cells using immunomagentic beads without the use of flow cytometry.

Embodiments further relate to methods and system that can replace other forms of cell marking such as, for example, Green Florescent Protein.

### BACKGROUND

The rapidly mutating nature of Human Immunodeficiency Virus (HIV), or other such viruses, and the adverse effects associated with routine drug therapy have necessitated the development of alternative therapeutic interventions. One of the obvious alternatives to chemotherapy is gene therapy. Advances in human CD34+ stem cell transplantation have prompted a search for new and potent gene therapy targets for suppression of HIV replication.

Advances in hematopoietic stem cell transplantation have renewed interest in anti-HIV gene therapy. Theoretically, modifying stem cells by insertion of an anti-HIV gene or gene-editing technologies can result in an HIV-proof immune system in patients after stem cell transplantation. The success of the “Berlin patient” receiving CCR5 negative cells for transplantation has demonstrated the feasibility of this approach.

Hence, there is an urgent need to develop new gene therapy vectors and targets that can be used to modify Hematopoietic stem cells in order to generate an HIV proof Immune response. Likewise, there is a need to develop methodologies that can be used to enrich vector-modified stem cells before transplantation in humans.

### BRIEF SUMMARY

The following summary is provided to facilitate an understanding of some of the innovative features unique to the embodiments disclosed and is not intended to be a full description. A full appreciation of the various aspects of the embodiments can be gained by taking the entire specification, claims, drawings, and abstract as a whole.

It is, therefore, one aspect of the disclosed embodiments to provide a method and system for treating a patient with virus.

It is another aspect of the disclosed embodiments to provide a method and system for treating a patient with HIV.

It is another aspect of the disclosed embodiments to provide a method and system for a vector for treating a patient with HIV.

It is another aspect of the disclosed embodiments to provide an enhanced method and system for a creating modified CD4 molecules.

It is yet another aspect of the disclosed embodiments to provide selectable stem cell modification for gene therapy using transient cell surface marking of modified cells using modified CD4 molecules.

The aforementioned aspects and other objectives and advantages can now be achieved as described herein. In an embodiment, a gene therapy method comprises modifying at least one CD4 molecule to form CD4mod, marking target cells with immunomagnetic beads, and killing the marked target cells. The method further comprises applying a vector that expresses TKSR39. The TKSR39 rapidly kills cells in a presence of Ganciclovir.

In an embodiment, the method can further include applying a second vector that expresses HIV Tat and a CRISPR-CCR5 cassette to knockout CCR5. The vector is expressed only in infected cells and kills the infected cells only in the presence of Ganciclovir. The SR39 mutant can comprise TK SR39.

In another embodiment, a method for selectable stem cell modification comprises introducing at least one transducing cell with a CD4mod molecule and transfecting cells with a CD4mod molecule, incubating the transduced cells with a plurality of magnetic beads, and magnetically sorting the transduced cells. The method further comprises applying a Lenti Tat CRISPR CCR5 to the transduced cells. Applying the Lenti Tat CRISPR CCR5 to the transduced cells creates expression of CD4. The CD4mod molecule can comprise an SR39 CD4mod molecule. In an embodiment, the method can further comprise allowing approximately 24 hours of incubation after transducing the cells with the CD4mod molecule.

The magnetic beads can comprise superparamagnetic beads. The method can further comprise covalently coating the superparamagnetic beads with a monoclonal antibody. Magnetically sorting the transduced cells further comprises attracting cells with the superparamagentic beads to a magnet.

In another embodiment, a method for developing a CD4mod molecule comprises synthesizing a full version of CD4 using RNA derived from cells, cloning the full version of CD4, truncating the cloned version of CD4, and generating CD4mod with mutated Env binding domains. The RNA derived from cells comprises Jurkat cell RNA. In an embodiment cloning the full version of CD4 comprises cloning the RNA derived from the Jurkat cells via RT-PCR.

In an embodiment, the truncated version of CD4 comprises a CD4 molecule without a cytoplasmic tail.

In an embodiment, generating CD4mod with mutated Env binding domains comprises mutating at least one amino acid in the truncated CD4 gene. Mutating the at least one amino acid in the truncated CD4 gene is achieved using site directed mutagenesis.

### BRIEF DESCRIPTION OF THE FIGURES

The accompanying figures, in which like reference numerals refer to identical or functionally-similar elements throughout the separate views and which are incorporated in and form a part of the specification, further illustrate the embodiments and, together with the detailed description, serve to explain the embodiments disclosed herein.

FIG. 1 depicts a flow chart of steps associated with a method for combining CCR5 knockout with TK-SR39 in accordance with the disclosed embodiments;

FIG. 2 depicts a flow chart of steps associated with a method for selecting and enriching modified CD34+ hematopoietic stem cells in accordance with the disclosed embodiments;

FIG. 3 depicts diagrams of modified CD4 molecules, in accordance with the disclosed embodiments;

FIG. 4 illustrates a flow chart of steps associated with a method for the development the CD4mod molecule for immunomagnetic bead-based purification of transduced stem cells, in accordance with an embodiment;

FIG. 5 depicts a table of sequences, in accordance with the disclosed embodiments;

FIG. 6 depicts a chart of CD4 mutants after transfection followed by flow cytometry analysis, illustrating CD4 expression results for all the mutant CD4 forms, in accordance with the disclosed embodiments;

FIG. 7 depicts a chart illustrating mutant forms of CD4 Trunc molecule fail to act as receptors for HIV Env, preventing virus entry, in accordance with the disclosed embodiments;

FIG. 8 depicts a flow chart of steps associated with a method for transduction and immunomagnetic bead-based sorting, in accordance with an embodiment;

FIG. 9 depicts diagrams of TK-SR39 vectors, in accordance with the disclosed embodiments; and

FIG. 10 depicts a chart illustrating knockout of CCR5 can be achieved in the TZM-TK-SR39 cell line while marking the cells with GFP, in accordance with the disclosed embodiments.

### DETAILED DESCRIPTION

The particular values and configurations discussed in these non-limiting examples can be varied and are cited merely to illustrate one or more embodiments and are not intended to limit the scope thereof.

The embodiments will now be described more fully hereinafter with reference to the accompanying drawings, in which illustrative embodiments of the invention are shown. The embodiments disclosed herein can be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout. As used herein, the term “and/or” includes any and all combinations of one or more of the associated listed items.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The methods and systems disclosed herein provide improvements to U.S. patent application Ser. No. 15/329,318 titled “Conditional Cytotoxic Gene Therapy Vector for Selectable Stem Cell Modification for Anti HIV Gene Therapy.” patent application Ser. No. 15/329,318 is herein incorporated by reference in its entirety.

The present methods and systems use CD4 as a cell surface marker. While green fluorescent protein (GFP) can be used as the cell marker it can also elicit an immune response making it less effective. Therefore, CD4 is a better marker and can be used in vivo. Modification of the CD4 molecule into CD4mod is disclosed in an embodiment, along with vectors associated with the modification of CD4 (instead of CD4 delta CD). In certain embodiments, the utility of CD4mod extends beyond anti-HIV gene therapy to any vector that requires purification of cells.

Hematopoietic stem cells represent an ideal target for HIV (or other such viruses) gene therapy as they produce all the cells involved in HIV pathogenesis. Lentiviral vectors are well suited for introducing a gene of interest in stem cells as they have the potential to transduce a high percentage of stem cells without loss of “sternness”. Transiently marking the cells for GFP expression is thus feasible. While use of GFP based sorting to enrich vector transduced cells is acceptable for in vitro studies, its use in vivo may be a concern due to unwanted immune responses against GFP expressing cells, leading to their elimination by the immune system. Furthermore selection of cells via flow cytometry subjects the cells to extensive manipulation that, in the case of stem cells, may reduce both viability and stemness for reconstitution. Hence, an alternate strategy, as disclosed herein is to develop CD4mod as a cell surface marker to purify modified stem cells using immunomagentic beads. Besides providing a platform for cell selection, CD4mod has added benefits as it can be expressed only on HIV infected or tat transduced cells.

In the embodiments disclosed herein, new gene therapy vectors and targets that can be used to modify Hematopoietic stem cells in order to generate an HIV proof Immune response are disclosed. Such therapies necessarily include multiple layers of safety as well as multiple targets to attack the virus at various fronts. The embodiments disclosed herein also provide methodologies that can be used to enrich vector modified stem cells before transplantation in humans.

In an exemplary embodiment, a two-step vector system that incorporates multiple safety features, as well as a selection feature can be used. The first vector, vector 1, is a conditional vector that only expresses genes of interest in the presence of the HIV Tat protein. This vector will express TKSR39, a TK mutant that is highly potent at killing cells in the presence of small amounts of Ganciclovir. Ganciclovir is a drug routinely used for Herpes virus treatment in HIV infected individuals. Moreover, the vector incorporates an added unique feature in that it also has a Tat inducible CD4mod (CD4 lacking a cytoplasmic domain as well as HIV Env binding region) that can provide a mechanism to purify the cells using immunomagentic beads.

To induce a transient expression of CD4mod a second vector, vector 2, is used that expresses HIV Tat and a CRISPR-CCR5 cassette to knockout CCR5. Hence, cells modified by this two-step vector system can have CCR5 knockout as well as conditional expression of TKSR39 and CD4mod that can allow easy selection of cells. This method will allow for enrichment of vector modified stem cells before in vivo transplantation leading to better engraftment while providing multiple layers of anti HIV activity. It should be appreciated that while the methods and systems described herein refer to treatment of HIV, it should be appreciated that the methods and systems may generally be applied to other such viruses.

In an embodiment, a method is disclosed that replaces Green Fluorescent Protein (GFP) based selection with a cell surface marker allowing immunomagnetic bead-based sorting. The GFP can be replaced with an endogenous cell surface marker to eliminate an unwanted immune response and allow for immunomagnetic bead based sorting. The embodiments disclosed herein use modified CD4 molecules (CD4mod) lacking the cytoplasmic domain and the HIV Env binding region. This selection marker can be transiently expressed only in the presence of Tat. This allows for the optimization of the immunomagnetic bead based purification of cells. The vector does not exhibit leaky Tat expression, hence unwanted bystander effects are not a concern.

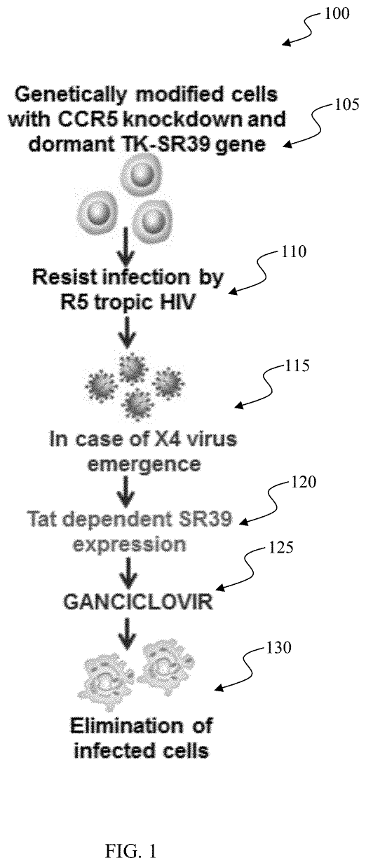

An exemplary embodiment of a method 100 for combining CCR5 knockout with TK-SR is illustrated in FIG. 1. CCR5 is a co-receptor for HIV entry and is predominantly used during initial establishment of infection. Interestingly, HIV can use another co-receptor, CXCR4, which is utilized by viruses later during the infection and is associated with a rapid decline in CD4 cells. This is the mechanism that ultimately leads to the development of AIDS. In the absence of CCR5, HIV can switch co-receptor usage to CXCR4, resulting in the rapid development of AIDS. Thus, gene therapy strategies targeting CCR5 alone lack wide spread applicability and are less likely to be successful.

In the embodiments disclosed herein, a gene therapy approach is applied wherein CCR5 knock-out is combined with a conditional cytotoxic gene TK-SR39. At step 105, genetically modified cells with CCR5 knockdown and a dormant TK-SR39 gene are introduced, as illustrated. As shown at step 110, these cells resist infection by R5 tropic HIV. Step 115 illustrates a mechanism that can be included in case of X4 virus emergence. The mechanism of inhibition of CXCR4 tropic viruses is the specific killing of infected cells by a TKSR39 and Ganciclovir combination. At step 120, Tat dependent SR39 expression is shown. It is important to note that SR39 expression only occurs upon virus infection or Tat expression. At this point, Ganciclovir can be provided, as shown at step 125, which, in the presence of TK-SR39, is converted into a toxic product and results in the elimination of infected cells as illustrated at step 130. Thus, this dual approach will be broadly effective against both CCR5 and CXCR4 tropic HIV isolates.

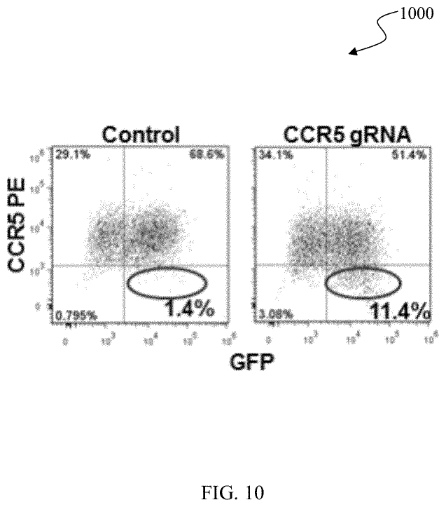

CCR5 knockout using Lenti-Tat-CRISPR-CCR5 has been tested. Specifically, tests have been conducted to verify the efficacy of the methods and systems disclosed herein. Data shows that knockout of CCR5 can be achieved in the TZM-TK-SR39 cell line while marking the cells with GFP. This is illustrated as illustrated in chart 1000 provided in FIG. 10. To further validate the vector, a T7 endonuclease 1 assay can be used to determine the efficiency of CCR5 gene knockout. For this, a PCR using primers on either side of the crRNA target sequence followed by T7 endonuclease 1 digest can be used. The efficiency of the embodiments can be tested in both TZM and CEM-CCR5 cell lines.

HIV gene therapy approaches targeting CCR5 alone bear the caveat that emergence of more pathogenic CXCR4 utilizing viral variants is expected. The disclosed embodiments, which include combining CCR5 knockout with a cytotoxic gene, provides another layer of anti-HIV activity and a necessary fall back mechanism in case of CXCR4 tropic virus emergence. Thus, the embodiments are effective against diverse HIV isolates.

In certain embodiments, the TK-SR39 based cytotoxic approach is targeted towards eliminating HIV infected cells rather than targeting a viral protein. Development of resistance to the cytotoxic TK based vector is hence unlikely.

As Tat is an early viral protein, the systems and methods disclosed herein target a cell for destruction before millions of virus progeny are generated. This can effectively limit virus replication as well as establishment of new latent reservoirs. Using TK-SR39 as disclosed herein, for selectively killing and eliminating virus infected cells is also a viable way to suppress the virus.

The disclosed embodiments are thus directed to the ability to select/enrich vector modified stem cells using Tat mediated transient expression of CD4mod. By utilizing a cell surface marker cells are enriched via immunomagnetic beads, which provides a means for applying this therapy in clinical use.

Ganciclovir dependent cytotoxicity of TK-SR39 provides an additional layer of safety in case of virus co-receptor switching. Ganciclovir is approved for treatment of cytomegalovirus retinitis in HIV patients and has an impeccable safety record with regards to long term administration. The concentrations necessary for the embodiments disclosed herein are in line with those achievable in vivo.

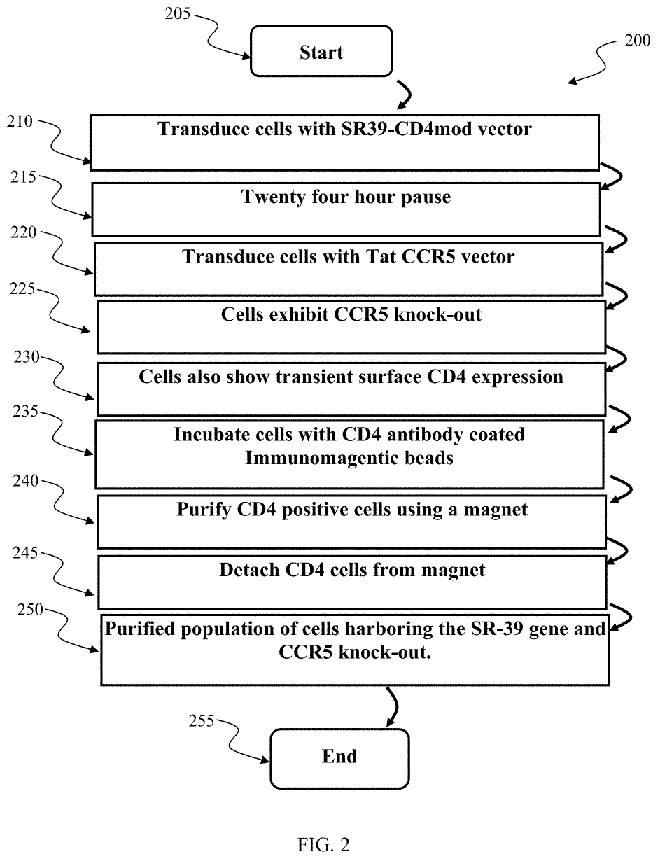

Other embodiments disclosed herein are intended to provide an immunomagnetic bead based method for selecting and enriching modified CD34+ hematopoietic stem cells. FIG. 2 illustrates such a method 200. The method starts at step 205. At step 210, cells can be transduced with the SR39-CD4mod vector. In some embodiments, an approximate 24-hour delay is necessary, as illustrated at step 215. The delay is necessary because the SR39-CD4mod vector is an integrating vector, while the Tat CCR5 vector is non-integrating vector. If both vectors are added at the same time it could potentially lead to integration of the Tat-CCR5 vector which is not desirable. In certain embodiments other amounts of time may be required.

Next, at step 220, cells can be transduced with a Tat CCR5 vector. This non-integrating vector will transiently produce Tat protein that drives the expression of CD4mod from the SR39-CD4mod vector and at the same time knockout CCR5 by the CCR5 CRISPR cassette. The cells exhibit CCR5 knockout as illustrated at step 225. In addition, as illustrated at step 230, the cells also show transient surface CD4mod expression. The transient nature of the surface expression is important. At step 235, the cells can be incubated with CD4 antibody coated Immunomagnetic beads. The antibodies on the beads bind to antigens on the cells surface. A magnet can then be used to purify (or select) the CD4mod positive cells as illustrated at step 240. The CD4mod cells are then detached from the magnet as illustrated at step 245, resulting in a purified population of cells harboring the SR-39 gene with CCR5 knockout, as illustrated at step 250. The purified population of cells is thus ready for application in gene therapy treatment of, for example, HIV. The method ends at step 255.

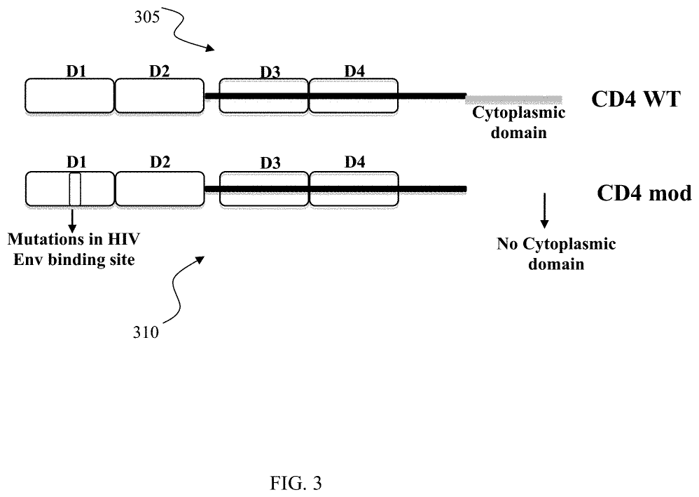

As noted, use of GFP based sorting to enrich vector transduced cells is acceptable in vitro. However, its use in vivo may be undesirable because of an unwanted immune response against GFP. Thus, in another embodiment a modified CD4 molecule 310 shown in FIG. 3 (CD4mod) can be used as a cell surface marker to purify modified stem cells using immunomagnetic beads thereby eliminating use of flow cytometry. CD4mod is ideal for a number of reasons. First, undifferentiated CD34+ stem cells do not express CD4 305 (illustrated in FIG. 3). Further, the vector expresses CD4mod 310 only transiently in the presence of Tat. Also, HIV infected cells are CD4+ and hence the marker will not interfere with immune function. CD4mod 310 lacks a cytoplasmic domain preventing any downstream signaling. And finally, CD4mod 310 also lacks specific amino acids involved in Env binding; hence any leaky expression will not generate additional targets for HIV infection.

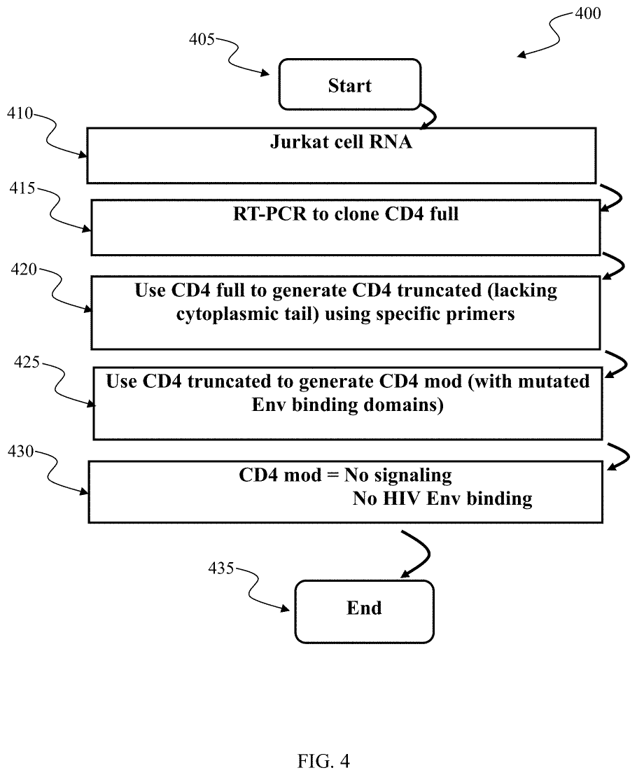

To develop the CD4mod molecule 310 for immunomagnetic bead-based purification of transduced stem cells, a stepwise design approach can be employed as illustrated by flow chart 400 provided in FIG. 4. It should be appreciated that CD4mod is a molecule that can be used for sorting any transduced or transfected cells and can have uses beyond HIV gene therapy treatment. For example, in some non-exclusive embodiments, the methods and systems disclosed herein can be used in laboratory research, providing researchers the ability to purify transfected or transduced cells. In such cases, the CD4mod can be incorporated into such vectors and used for sorting as disclosed herein.

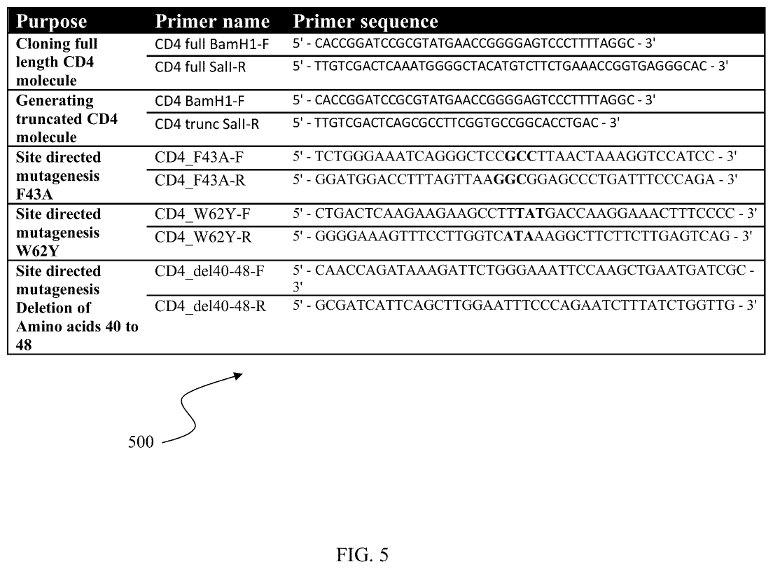

The stepwise design approach begins at block 405. Block 410 illustrates the starting Jurkat cell RNA. Next, a full version of CD4 using RNA derived from Jurkat cells can be cloned via RT-PCR, as illustrated at block 415. Using CD4 full as a template and with specific primers, a truncated CD4 molecule lacking the cytoplasmic tail can be generated, as illustrated at block 420. The primers are used for PCR based strategies to generate constructs as well as induce mutations. FIG. 5 illustrates a table 500 with the actual sequences.

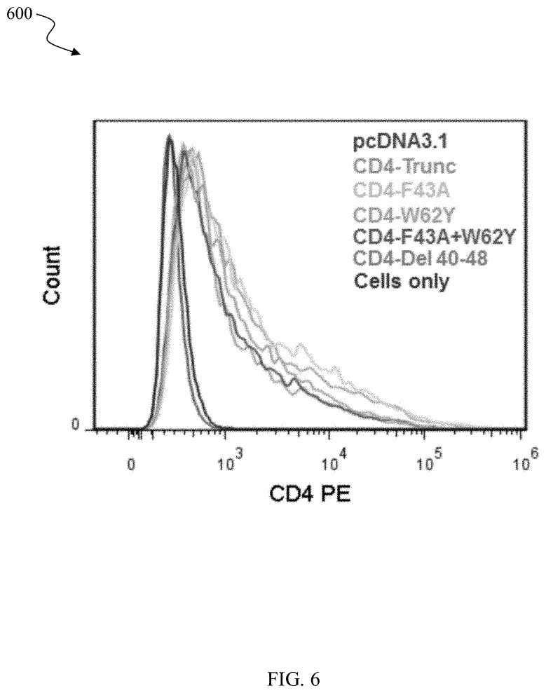

Once the truncated CD4 molecule has been developed, it can then be used to generate CD4 mod with mutated Env binding domains as shown at block 425. To generate CD4mod specific amino acids in the truncated CD4 gene are mutated using site directed mutagenesis. Phe at position 43 (F43) and Trp at position 62 (W62) in CD4 are critical for HIV Env binding. Also, the major contact region of HIV Env with CD4 is in the amino acid 40-48 region of CD4. Hence, mutations in the CD4-Trunc molecule, including F43A, W62Y, and double mutant F43A+W62Y, can be made via site directed mutagenesis. In addition, CD4-Trunc deletion mutant 40-48Del lacking amino acids 40-48 of CD4 can be created via site directed mutagensis. The cell surface expression of the truncated CD4, along with different CD4 mutants can be confirmed by flow cytometry analysis. Thus, as illustrated at block 430, CD4mod does not facilitate signaling and has no HIV Env binding. The method ends at block 435. FIG. 6 illustrates a chart 600 of CD4 mutants after transfection followed by flow cytometry analysis, illustrating CD4 expression results for all the mutant CD4 forms.

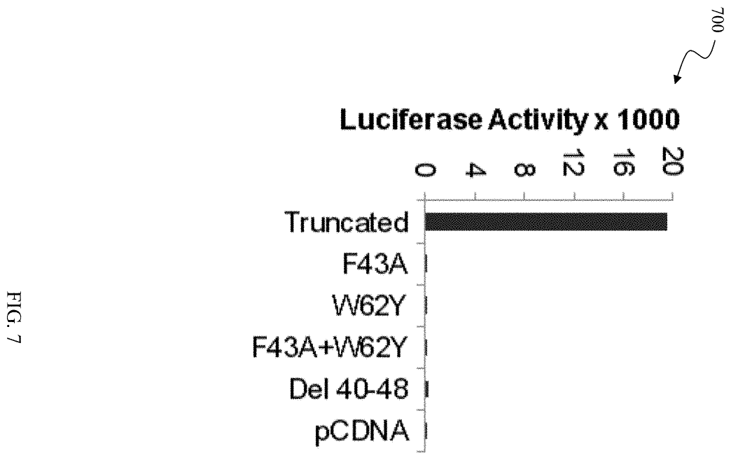

Testing of the capability of the CD4-WT, CD4-Trunc, and various CD4 mutants (CD4mod) to act as a receptor for HIV have been conducted. For such tests HeLa cells can be transfected with various CD4 constructs followed by infection with Lai Env pseudotyped reporter virus particles. HeLa cells express CXCR4 endogenously and addition of CD4 makes these cells susceptible to infection with an X4 tropic virus like Lai. CD4-trunc acts as a receptor for HIV infection while the CD4 mutants (CD4mod) failed to act as a receptor as evident by lack of virus entry in their presence. FIG. 7 illustrates a chart 700 that shows that mutant forms of CD4 Trunc molecule fail to act as receptors for HIV Env, preventing virus entry.

The methods and systems disclosed herein have many advantages over current techniques. The construct is a conditional vector so it only expresses in infected cells or after introduction of Tat for transient expression and sorting. CD4 is an ideal molecule for marking HIV infected cells because it is expressed on infected cells. Anti CD4 immunomagnetic beads are readily available and are routinely used for purification of CD4+ cells in laboratories across the world. CD4mod lacks a cytoplasmic domain preventing any downstream signaling. CD4mod also lacks specific amino acids involved in Env binding (38, 39), hence any leaky expression will not generate additional targets for HIV infection. More importantly the use of CD4mod can be expanded to all those methods where cell purification is needed after genetic modification of cells via transfection or transduction. CD4mod can be a marker used in any vector where cell purification is needed.

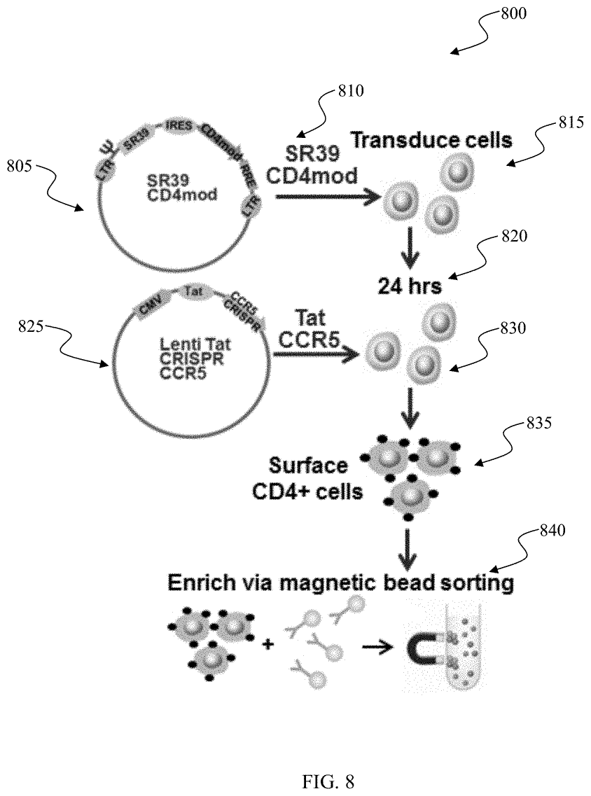

The FIG. 8 illustrates a method 800 for selectable stem cell modification for gene therapy using transient cell surface marking of modified cells using modified CD4 molecules. At step 805, an SR39 CD4mod molecule is shown. At step 810, the SR39 CD4mod is applied to transduce cells, as shown at step 815. The combination is given 24 hours as shown by step 820, before a Lenti Tat CRISPR CCR5, as shown by step 825, is applied to the cells as shown at step 830. Step 835 illustrates the surface CD4 expression on the cells.

At step 840, enrichment via bead sorting is applied. The process of bead-based sorting provides an easy alternative to use of flow cytometry. Flow cytometry requires the use of expensive equipment, expertise and a process where the cells have to undergo extensive manipulation and stress. Bead-based sorting can be used for cell surface expressed makers if an antibody is capable of binding to the marker and magnetic beads. In certain embodiments, superparamagnetic beads (4.5 μm diameter, or other diameters according to design considerations) are covalently coated with a monoclonal antibody specific for CD4. The beads are incubated with transduced cells expressing the CD4 molecule. The cell surface CD4 molecule binds to the CD4 antibody coated on magnetic beads which can then be purified by applying a magnetic force via a simple magnet. As a result, the cells purified using immunomagnetic bead based sorting show CCR5 knock-out with combined expression of TK-SR39. While CCR5 knock-out is permanent, TK gene and CD4 expression is transient and dependent on the presence of Tat or HIV infection.

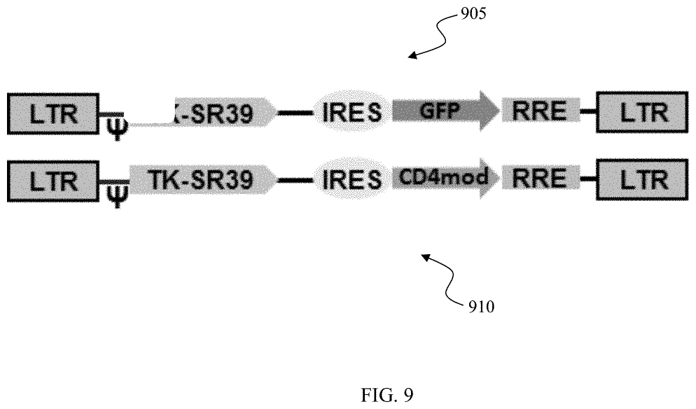

FIG. 9 illustrates a vector design using GFP at 905 and CD4Mod at 910. The vector comprises an LTR based promoter at the 5′ end followed by a packaging signal that allows production of lentiviral particles. Immediately 3′ to the packaging signal is the TK-SR39 gene whose expression would be Tat dependent due to being under the control of LTR. The Internal Ribosomal Entry Site (IRES), immediately upstream of the GFP/CD4mod molecules, allows for GFP/CD4 expression in conjunction with the TK-SR39 gene. Thus, cells transduced with the vector express TK-SR39 and CD4mod proteins only in the presence of Tat or HIV infection.

Based on the foregoing, it can be appreciated that a number of embodiments, preferred and alternative, are disclosed herein. For example, in an embodiment, a gene therapy method comprises modifying at least one CD4 molecule to form CD4mod, marking target cells with immunomagnetic beads, and killing the marked target cells. In an embodiment the method comprises applying a first vector that expresses TK-SR39. In an embodiment the method comprises applying a second vector that expresses HIV Tat and a CRISPR-CCR5 cassette to knockout CCR5.

In an embodiment, the TK-SR39 rapidly kills cells in a presence of Ganciclovir. In an embodiment the vector is expressed only in infected target cells and kills the infected target cells only in the presence of Ganciclovir. In an embodiment the SR39 mutant comprises TK-SR39.

In an embodiment, a method comprises transducing cells with a CD4mod molecule and transfecting at least one cell with a CD4mod molecule, incubating the transduced cells with a plurality of magnetic beads and magnetically sorting the transduced cells. In an embodiment the method further comprises applying a Lenti Tat CRISPR CCR5 to the transduced cells. In an embodiment the CD4mod molecule comprises an SR39 CD4mod molecule.

In an embodiment, the method further comprises allowing incubation after transducing the cells with the CD4mod molecule. In an embodiment applying a Lenti Tat CRISPR CCR5 to the transduced cells creates expression of CD4.

In an embodiment, the plurality of magnetic beads comprises a plurality of superparamagnetic beads. In an embodiment the method further comprises covalently coating the plurality of superparamagnetic beads with a monoclonal antibody. Magnetically sorting the transduced cells further comprises attracting cells with the superparamagentic beads to a magnet.

In an embodiment, a method for developing a CD4mod molecule comprises synthesizing a full version of CD4 using RNA derived from cells, cloning the full version of CD4, truncating the cloned version of CD4, and generating CD4mod with mutated Env binding domains.

In an embodiment, RNA derived from cells comprises Jurkat cell RNA. Cloning the full version of CD4 comprises cloning the RNA derived from the Jurkat cells via RT-PCR.

In an embodiment, the truncated version of cloned CD4 comprises a CD4 molecule without a cytoplasmic tail.

In an embodiment, generating CD4mod with mutated Env binding domains comprises mutating at least one amino acid in the truncated CD4 gene. Mutating the at least one amino acid in the truncated CD4 gene is achieved using site directed mutagenesis.

It will be appreciated that variations of the above-disclosed and other features and functions, or alternatives thereof, may be desirably combined into many other different systems or applications. Furthermore, it can be appreciated that various presently unforeseen or unanticipated alternatives, modifications, variations or improvements therein may be subsequently made by those skilled in the art which are also intended to be encompassed by the following claims.

What is claimed is:

1. A gene therapy method comprising:
modifying at least one CD4 molecule to form CD4mod;
marking target cells with immunomagnetic beads; and
killing said marked target cells.

2. The method of claim 1 further comprising:
applying a first vector that expresses TK-SR39.

3. The method of claim 1 further comprising:
applying a second vector that expresses HIV Tat and a CRISPR-CCR5 cassette to knockout CCR5.

4. The method of claim 2 wherein said TK-SR39 rapidly kills cells in a presence of Ganciclovir.

5. The method of claim 4 wherein said vector is expressed only in infected target cells and kills said infected target cells only in said presence of Ganciclovir.

6. The vector of claim 3 wherein an SR39 mutant comprises TK-SR39.

7. A method comprising:
transducing cells with a CD4mod molecule and transfecting at least one cell with a CD4mod molecule;
incubating said transduced cells with a plurality of magnetic beads; and
magnetically sorting said transduced cells.

8. The method of claim 7 further comprising:
applying a Lenti Tat CRISPR CCR5 to said transduced cells.

9. The method of claim 8 wherein said CD4mod molecule comprises an SR39 CD4mod molecule.

10. The method of claim 8 further comprising:
allowing incubation after transducing said cells with said CD4mod molecule.

11. The method of claim 8 wherein applying a Lenti Tat CRISPR CCR5 to said transduced cells creates expression of CD4.

12. The method of claim 8 wherein said plurality of magnetic beads comprise a plurality of superparamagnetic beads.

13. The method of claim 12 further comprising:
covalently coating said plurality of superparamagnetic beads with a monoclonal antibody.

14. The method of claim 13 wherein magnetically sorting said transduced cells further comprises:
attracting cells with said superparamagentic beads to a magnet.

15. A method for developing a CD4mod molecule said method comprising:
synthesizing a full version of CD4 using RNA derived from cells;
cloning said full version of CD4;
truncating said cloned version of CD4; and
generating CD4mod with mutated Env binding domains.

16. The method of claim 15 wherein said RNA derived from cells comprises Jurkat cell RNA.

17. The method of claim 16 wherein cloning said full version of CD4 comprises:
cloning said RNA derived from said Jurkat cells via RT-PCR.

18. The method of claim 15 wherein said truncated version of cloned CD4 comprises a CD4 molecule without a cytoplasmic tail.

19. The method of claim 15 wherein generating CD4mod with mutated Env binding domains comprises:
mutating at least one amino acid in said truncated CD4 gene.

20. The method of claim 19 wherein mutating said at least one amino acid in said truncated CD4 gene is achieved using site directed mutagenesis.
